(19) United States
(12) Patent Application Publication
(10) Pub. No.: US 2014/0364959 A1
(43) Pub. Date: Dec. 11, 2014

(54) STENT PROSTHESIS INTENDED TO BE IMPLANTED IN THE DIGESTIVE TRACT OF A PATIENT

(71) Applicant: ASSISTANCE PUBLIQUE - HOPITAUX DE PARIS (APHP) -, PARIS CEDEX 10 (FR)

(72) Inventors: Alain Attar, Paris (FR); Yoram Bouhnik, Asnieres Sur Seine (FR)

(21) Appl. No.: 14/368,155

(22) PCT Filed: Dec. 24, 2012
(86) PCT No.: PCT/EP2012/076864

§ 371 Date: Jun. 23, 2014

(30) Foreign Application Priority Data
Dec. 23, 2011 (FR) 1162442

(51) Int. Cl. A61F 2/04; A61F 2/90

(52) U.S. Cl. CPC A61F 2/04; A61F 2/90; A61F 2002/045; A61F 2002/044; A61F 2250/0098
USPC 623/23.7

(57) ABSTRACT

The invention relates to a prosthesis that is compressible and expandable in a radial direction, intended to be implanted in the digestive tract of a patient. According to the invention, such a prosthesis comprises: a downstream conical collar having end diameter D3, a main tabular body having diameter D2, an upstream conical collar having end diameter D1, said upstream collar not being covered by any material and having an end diameter D1 greater than the diameter D2 of said main body and greater than the end diameter D3 of said downstream collar, and said downstream collar being fully or partially covered by at least one polymer material.

### 1. FIELD OF THE INVENTION

The field of the invention is that of the design and production of prostheses for medical and/or surgical use.

More precisely, the invention relates to an expandable prosthesis intended to be implanted in the digestive tract of a patient. The digestive tract is constituted by all of the hollow organs that form the transit of the food from the oral cavity to the rectum, which includes the oesophagus, the stomach, the duodenum, the jejunum, the ileum, the caecum, the ascending colon, the transverse colon, the descending and sigmoid colon, the rectum.

This type of prosthesis is intended to be implanted for the treatment of certain digestive pathologies, and in particular short digestive stenoses, such as those occurring in the framework of cancer, or Crohn's disease.

### 2. PRIOR ART

Crohn's disease is a chronic inflammatory bowel disease, probably of auto-immune origin. This affection manifests itself by inflammatory lesions, along with cavitating and superficial ulcerations, or even fractures of the membrane, in particular on the colon and the ileum. The demographic data of the French national cohort CESAME (2004-2005) reveals that the small intestine is affected in 70% of patients afflicted with Crohn's disease. Although drug treatments exist, recourse to surgery to remove the ulcerated intestine fragments is practically the rule since 70% undergo surgery. After a resection, an endoscopic recurrence occurs in 90% of cases and can in time result in the appearance of a stenosis.

Stenosis is an anatomical modification that results in the narrowing of a tubular structure. It therefore results in a total or partial obstruction of the hollow organ affected.

When the stenosis affects the digestive tract, the obstruction of the digestive tracts results in a slowing down of the intestinal transit, along with pain, and the whole can result in an alteration in the general condition, poor absorption of food and even complete intestinal obstruction.

Stenoses can be classified according to their length. Stenoses referred to as “short” can then be distinguished, of which the length is less than 4 to 5 cm, and long stenoses, of which the length is greater than 4 to 5 cm. Long stenoses require recourse to surgery in order to remove them. On the other hand, short intestinal stenoses can be treated either by surgery, or by hydrostatic endoscopic dilatation.

Hydrostatic endoscopic dilatation consists in passing a catheter in the lumen of the intestine of the sedated patient. The catheter contains a balloon which is progressively inflated with water at the level of the stenosis, which allows for its dilatation under endoscopic and radiological control. The lumen of the digestive tract opens and the stenosis is as such treated. The balloon is then removed. However, hydrostatic endoscopic dilatation has a complication rate between 1% and 10% (average 2%, Hassan 2007), and a risk of recurrence of 30% to 50%. In the latter case, the patient then undergoes another endoscopic dilatation, or even surgery.

In order to improve the efficacy of the treatment of short stenoses and reduce the risks of recurrence without having recourse to surgery, metal expandable tubular prostheses have been proposed. These expandable prostheses have the form of tubes with a mesh structure, i.e. comprised of interlaces of wire, generally of a metal nature, forming meshes in the manner of a wire mesh. Most of these prostheses have the form of a main tubular body extended at each of its ends by a frustoconical portion forming a flared collar. These collars, located on either side of the main body, have an end diameter that is wider than that of the main body. On the other hand, the collars are rigorously identical: they have, between them, the same shape and the same diameter. The main objective of these collars is to enable a flaring of the ends of the prosthesis in order to make it possible to more easily pass an endoscope through the prosthesis in place if needed, for example in order to explore the upstream digestive tract.

Digestive prostheses are compressed in a catheter in order to be conveyed to the stenosis under endoscopic control. The catheter containing them is then withdrawn while still leaving the prosthesis in place. This withdrawal enables the prosthesis to be deployed in the digestive tract, at the level of the stenosis, and to very quickly restore the diameter of the tract and the circulation of the alimentary bolus.

Historically, the first expandable metal prostheses were not covered with any polymer film. The main therapeutic indication of these prostheses was the palliative treatment of short stenoses in patients suffering from colorectal cancer. Indeed, the tumour growth finishes by obstructing the intestinal lumen, as such weakening the patient and causing strong pain. The metal nature of these prostheses, without any portion covered by any material, allows them to incarcerate into the digestive tissue. More precisely, the tissue, or the tumour, develops around the meshes of the prosthesis. The prosthesis is then embedded in the digestive tissue and as such offers sustainable relief for the patient until death. In this case, the prosthesis is never extracted. The intraprosthetic implanting of another prosthesis is then sometimes necessary due to tumour growth.

More recently, other types of prostheses have been developed in order to care for short stenoses with the goal of healing, and not only for palliative purposes.

These prostheses are then made of metal entirely covered by a polymer film, in order to prevent them from being incarcerated and allow them to be withdrawn. Covering the meshing structure of the prosthesis with a polymer film makes it possible to remove it more easily. However, this system has other disadvantages.

Indeed, these prostheses are often spontaneously and very rapidly eliminated by the patient by natural means. The intestinal peristalsis and the pressure exerted by the alimentary bolus cause the early migration of these prostheses. Once eliminated or moved, these prostheses can no longer fulfil their function of dilatation of the stenosis. A Japanese team reported the case of patients for whom covered prostheses had been implanted for the treatment of stenoses of the anastomosis of ileo-colic resections consecutive to Crohn's disease. All of the patients eliminated their prostheses rectally (N. Matsuhashi et al., Gastrointestinal Endoscopy, 51 (3), 2000). Another study reports that the use of this type of prosthesis in the oesophagus made it possible to care for 56% of the patients. However, cases of early migration were observed in 36% of the patients (J. C. Bakken et al, Gastrointestinal Endoscopy, 72(4), 2010). This phenomenon of spontaneous and early migration was also observed in 22% of the patients treated for a stenosis consecutive to an obstructive colorectal cancer (G. Fernandez-Esparrach et al. The American Journal of Gastroenterology, 2010). Finally, a recent study of patients receiving intestine surgery for Crohn's disease and having anastomostic stenoses demonstrated that fully covered prostheses migrated early in 70% of the patients treated. Among these patients, one of them had to undergo endoscopic dilatation of the stenosis, in order to retrieve the prosthesis which had migrated early upstream of the stenosis. Another developed an abscess one month after the migration (Attar et al, Inflamm Bowel Dis, 2011, going to press).

The phenomenon of spontaneous migration of prostheses is not necessarily an obstacle in the immediate dilatation of the stenosis. It is however essential to control the duration of the dilatation in order to treat a stenosis with efficacy. However, spontaneous migration does not make it possible to control this parameter. It is therefore not possible, from a medical and an ethical standpoint, to implant a prosthesis of which the duration of the setting into place is not controlled and which is susceptible to cause complications (abscess, incarceration etc.).

Moreover, these prostheses are expensive and their expulsion by the body of the patient greatly limits the efficacy of their action. It is therefore necessary to design prostheses that solve in particular the problem of early migration of digestive prostheses.

### 3. OBJECTIVE OF THE INVENTION

The invention has in particular for objective to overcome these disadvantages of prior art.

More precisely, an objective of the invention is to provide, in at least one embodiment, a digestive prosthesis that makes it possible to treat short stenoses with efficacy.

The invention further has for objective to provide, in at least one embodiment, a prosthesis of which the use makes it possible to suppress or, at the very least, greatly limit the phenomenon of migration.

Another objective of the invention is to propose, in at least one embodiment, a prosthesis of which the use makes it possible to suppress or, at the very least, greatly limit the phenomenon of incarceration.

The invention also has for objective to provide, in at least one embodiment, a prosthesis that is simple to implant in order to limit the risks of perforation.

### 4. DISCLOSURE OF THE INVENTION

These objectives, as well as others that shall appear in what follows, are achieved using a prosthesis made from a base material, that is compressible and expandable in a radial direction, intended to be implanted in the digestive tract of a patient.

According to the invention, such a prosthesis comprises:
a downstream frustoconical collar having an end diameter D3,
a main tubular body having a diameter D2,
an upstream frustoconical collar having an end diameter D1,
   said upstream collar not being covered by any material and having an end diameter D1 greater than the diameter D2 of said main body and greater than the end diameter D3 of said downstream collar, and said downstream collar being covered or constituted, fully or partially, by at least one polymer material.

Note that, in the framework of this description, the terms “upstream” and “downstream” are defined in relation to the direction of migration of food in the digestive tract, i.e. from the oral cavity to the rectum.

Moreover, when reference is made to the diameters D1, D2 and D3 of the prosthesis, the latter must be understood as outside diameters of the prosthesis in its deployed state.

As such, the invention is based on an entirely original approach that consists in designing a prosthesis of which the dimensions of the collars are not exactly symmetrical on either side of the body of the prosthesis, contrary to the prostheses that are currently in use. Increasing the diameter of the upstream collar, in relation to prior art, constitutes in fact a mechanical brake. This brake allows the prosthesis to resist the thrust exerted on the one hand by the flow of food and liquids, and on the other hand by the contraction of the smooth muscles surrounding the digestive tract.

In terms of the invention, the upstream collar is never covered by a polymer film. The material constituting the prosthesis is therefore always left bare on the upstream collar. The absence of a covering material on the upstream collar contributes to a better maintaining of the prosthesis in place, on the stenosis, in order to accomplish its function of dilatation. Indeed, this absence of a covering allows the digestive tissue to invaginate through the meshes of the upstream collar. This phenomenon contributes in immobilising the prosthesis according to the invention on the stenosis. In addition, when the prosthesis is deployed in the digestive tract of the patient, the walls of the prosthesis are in contact with the mucous membrane. This contact causes forces of friction between the prosthesis and the wall of the digestive tract. These forces of friction make it possible to slow down the relative movement of the prosthesis in the digestive tract, and therefore to prevent, or at least greatly limit, the migration of the prosthesis within the digestive tract.

It is understood in the following description, that the expression “partially covered prosthesis” means that the prosthesis is covered, at least over a portion of its surface, with any polymer film.

Preferentially, the body of the prosthesis according to the invention has the shape of a cylinder of revolution.

Advantageously, the prosthesis according to the invention has a mesh structure.

The mesh structure allows the prosthesis to easily pass from a retracted position, necessary for implanting the prosthesis, to a functional deployed position. As such, the prosthesis is thrust against the inner wall of the digestive tract. The rapid deployment of the prosthesis makes it possible to immediately dilate the stenosis and to quickly re-establish the digestive transit. This mesh structure also allows for the compression of the prosthesis in order to introduce it into the catheter and allow it to be positioned without having recourse to surgery.

According to a first alternative, said base material is a metal material chosen from among stainless steel, titanium, chromium, nickel, cobalt or the combination of at least two of these materials, and said downstream collar is covered by at least one polymer material.

Preferably, the base material is then a nickel and titanium alloy such as nitinol. These materials have the advantage of being well tolerated by the patients. In addition, they are particularly resistant to the surrounding acid-base medium, and in constant contact with partially digested food. All the more so, these metals, or alloys, make it possible to produce shape memory materials. They are therefore particularly interesting for designing expandable and compressible prostheses. More particularly, thanks to this type of material combined with their mesh structure, the prostheses can be compressed in a catheter that is introduced through the natural cavities of the patient. These prostheses are delivered to the very location of the stenosis. The release of the prosthesis in the lumen of the digestive tract results in an immediate deployment of the latter. This deployment is authorised by the use of a shape memory material, which means that the prosthesis returns to its initial deployed form when the constraints exerted by the catheter disappear. In addition, the presence of a polymer material covering on the downstream collar makes it possible to facilitate the removal of the prosthesis. The absence of resistance during the removal facilitates not only the manipulation by the doctor, but also prevents injuring or irritating the digestive wall of the patient. It therefore makes it possible to limit the development of infections or perforations of the digestive wall, consecutive to the lesion caused by the forced removal of the prosthesis.

According to a second alternative of the invention, said base material constituting the prosthesis is a biodegradable polymer material, and said downstream collar is constituted of said polymer-base material.

As such, the prosthesis according to the invention can be fully constituted of a biodegradable polymer material. Using at least one biodegradable material to manufacture the prosthesis according to the invention makes it possible to design a digestive prosthesis which is resistant to the phenomenon of migration and which can also remain in the body of the patient until it is fully degraded. As such, the patient does not need a second endoscopy making it possible to extract the prosthesis from the digestive tract. It is indeed preferable to limit the number of endoscopies in the same patient. In addition to the cost of this act, endoscopy is not without risk. The perforation of the digestive wall by the endoscope is a risk to be taken into account. This risk is increased in patients of whom the digestive wall is weakened, which is in particular the case during a cancer, Crohn's disease and pathologies of the mucous membrane in general. A perforation is a surgical emergency. Certain research teams have begun to study oesophageal prostheses for treating benign stenoses (Y. Saito et al, World J Gastroenterology, 2007, 13(29)). However, a migration rate of about 77% was observed in the 13 patients treated and this, regardless of the aetiology of the stenosis.

Designing a biodegradable prosthesis furthermore makes it possible to resolve the problem of their management as contaminated medical waste, as well as that of their strong negative impact from an environmental standpoint.

Advantageously, said at least one biodegradable material is chosen from among polydioxanone (PDS), polylactic acid (PLA), poly-L-lactic acid (PLLA), polyglycolic acid (PGA), ε-caprolactone.

These polymer materials are particularly suited for the synthesis of flexible prostheses. They degrade in a period of a few weeks, with the period varying according to the pH and the nature of the enzymes present. The choice of the polymer to be implemented will therefore depend, among other parameters, on the anatomical destination of the prosthesis and of the chemical-physical conditions that it will have to support.

In a preferred embodiment, said at least one biodegradable polymer material is polydioxanone (PDS).

Advantageously, according to this second alternative of the invention according to which the base material of the prosthesis is a biodegradable polymer material, said mesh structure is carried out by a weaving or knitting of multifilament or monofilament threads of said at least one biodegradable polymer material.

This confers on the prosthesis great mechanical resistance which allows it to resist the constraints that is has to support after it is implanted, and in particular digestive peristalsis and the passage of the alimentary bolus.

In an advantageous embodiment, said main tubular body is covered, fully or partially, by at least one polymer material. The covering of the main body of the prosthesis according to the invention by at least one polymer material makes it possible to obstruct any fistulae that are produced on the stenosis. In addition, when the prosthesis according to the invention is made from a metal material, this makes it possible to facilitate the removal of the prosthesis once the stenosis is correctly dilated. The enlargement of the upstream collar then is sufficient to retain the prosthesis on the stenosis.

Preferably, said polymer material is chosen from the group comprised of polyurethane, polyvinyl chloride, urethane, silicone, polyamide, polyester, fluorine resin or the combination of at least two of these materials. Preferably, the polymer material that can cover the prosthesis according to the invention is silicone.

The presence of this layer has two main advantages:
prevent the irreversible adhesion of the prosthesis to the intestinal wall, by the invading of mesh of the prosthesis by the digestive tissue,
facilitate later the removal of the prosthesis from the body of the patient, without damaging the digestive wall in contact with the prosthesis.

As such, when the prosthesis is constituted of a metal material, it is particularly interesting to cover the downstream collar and the main body of the prosthesis with a polymer material such as silicone, fluorine resin, polyurethane, polyvinyl chloride, urethane, polyamide, polyester, or the combination of at least two of these materials. The presence of this covering on the main body and the downstream collar makes it possible indeed to facilitate the removal of the prosthesis, once the stenosis is dilated. This also makes it possible to seal any fistulae and to prevent the phenomenon of incarceration of the prosthesis in the tissue. As such, the practitioner is certain to not damage the inner wall of the digestive tract when he recovers the prosthesis by endoscopy. The covering of the body of the prosthesis, by one of these materials, is also interesting when the prosthesis is fully made from a biodegradable polymer material and the practitioner suspects or has detected the presence of fistulae on the stenosis.

Advantageously, the wall of said upstream collar according to the invention forms an angle alpha (α) between 30 degrees and 45 degrees in relation to the longitudinal axis of said prosthesis. Preferably, the wall of said upstream collar according to the invention forms an angle alpha (α) between 33 degrees and 45 degrees in relation to the longitudinal axis of said prosthesis.

The strong angulation of the upstream collar, in relation to prostheses of prior art, makes it possible to constitute a mechanical brake that is more substantial and more effective, without however damaging the inner wall of the digestive tract. In fact, the wall of the collars of current prostheses, and this for about twenty years, forms an angle between 15 degrees and 22 degrees with the body of the prosthesis. The value of these angles has always been limited in order to comply with the anatomical dimensions of the digestive tract of an adult. However, the inventors have discovered that a digestive prosthesis of which the wall of the upstream collar forms an angle between 30 and 45 degrees makes it possible not only to immobilise the prosthesis on the stenosis, but does not cause any more pain or lesions than current prostheses. The inventors have furthermore observed that when the prosthesis is made of a metal alloy with its main body and its downstream collar covered by a polymer material, the strong angulation of the upstream collar combined with its enlarged diameter is sufficient to retain the prosthesis on the stenosis and as such overcome the problem of migration of prostheses.

The prosthesis according to the invention has preferably a total length between 60 mm and 120 mm.

More preferably, the prosthesis comprises a total length between 60 mm and 80 mm.

These dimensions are sufficient for treating short stenoses, i.e. stenoses of which the length is less than or equal to 5 cm, that are obstructing the intestine. It is then interesting, during the placing of the prosthesis, to ensure that it symmetrically exceeds either side of the stenosis, if possible by at least 2 cm. The choice of the length of the prosthesis consequently depends on the length of the stenosis to be treated. Advantageously, the upstream collar has an end diameter D1 between 30 mm and 50 mm.

Preferably, said upstream collar has an end diameter D1 of 40 mm and an angle of 30 degrees.

These dimensions make it possible to design an upstream collar that effectively retains the prosthesis in the digestive tract, on the stenosis, without however injuring the digestive wall or oven inducing unpleasant or painful feelings for the patient. These dimensions are, moreover, compatible with both the introduction of the prosthesis according to the invention in a catheter, and the anatomical proportions of the digestive tract in adults.

According to an advantageous embodiment, said tubular body comprises a length between 20 and 80 mm and an outside diameter D2 between 15 and 25 mm.

Preferably, the length of said tubular body is between 24 mm and 60 mm, and its outside diameter D2 is equal to 20 mm. These dimensions are sufficient to dilate a short stenosis and re-establish the normal transit of food in the intestine.

Advantageously, said downstream collar has a length between 15 and 25 mm and an end diameter D3 between 25 mm and 32 mm.

Preferably, said downstream collar has a length of 18 mm and an end diameter D3 equal to 26 mm.

Such a collar participates in maintaining the prosthesis in the digestive tract. The flared shape of this collar, combined with its diameter which is slightly enlarged in relation to that of the main body, constitutes a second mechanical brake allowing the prosthesis according to the invention to resist the pressure exerted by the digestive peristalsis and/or the alimentary bolus.

Advantageously, the prosthesis according to the invention comprises at least one radiopaque marker. The inclusion of radiopaque markers, or radiological markers or, on the outside surface of the prosthesis allows for radiological locating and the verification of its positioning. These markers are released at the moment of the degradation of the prosthesis and are eliminated by the patient by natural means. These radiological markets can be chosen from among gold, platinum, iridium and any biocompatible metal that is opaque to X-rays.

Preferably, the prosthesis comprises two radiopaque markers on each collar and a radiopaque market on the longitudinal body. Radiological markers can be fixed on the prosthesis by any method well known to those skilled in the art.

More precisely, each of these radiopaque markers can be incorporated into the coating of the prosthesis according to the invention and/or be fixed onto the meshing of the prosthesis. Preferably, the marker is incorporated into the coating of polymer material, when this is possible.

In an advantageous embodiment, the prosthesis according to the invention is covered or includes in said polymer material at least one active ingredient. Preferably, said active ingredient is chosen from among an antibody, a statin, corticoids, an antifibrosant molecule and combinations of them.

The following examples of antibodies can be mentioned: infliximab, adalimumab, certolizumab, vedolizumab, ustekinumab, natalizumab. The following examples of antifibrosant molecule can also be mentioned: resveratrol, growth hormone, extracts of Scutelleria or of Boswellia, mitomycin-C, CTRP-3 (C1qTNG-related protein 3), anti-TGF-13, interleukin 10, TNRF2, PEG 15-20, biological films containing derivatives of hyaluronic acid such as chitosan-dextran and carboxymethyl-chitosan and Daikenchuto.

As such, it will be possible to treat the stenosis in a more targeted manner and to increase the therapeutic potential of the prosthesis.

The invention further has for object a method for treating benign stenoses in general, short (measuring less than 5-8 cm), colonic and intestinal anastomotic stenoses, postoperative stenoses with or without fistulae, and any neoplastic or non-neoplastic stenosis with fistula and this, regardless of the portion of the digestive tract, as long as they are accessible through digestive endoscopy.

### 5. LIST OF FIGURES

Other characteristics and advantages of the invention shall appear more clearly when reading the following description of preferred embodiments, given simply for the purposes of information and in a non-limiting manner, and of the annexed drawings, among which:

FIG. 1 shows a side view of a first embodiment of a prosthesis according to the invention, with the prosthesis being fully deployed;

FIG. 2 shows a perspective view of a second embodiment of a prosthesis according to the invention, with the prosthesis being fully deployed;

FIG. 3 shows a side view of a third embodiment of a prosthesis partially covered with a biodegradable material, with the prosthesis being fully deployed;

FIG. 4 shows a prosthesis according to the invention partially deployed, when it is being set into place for treating a stenosis thanks to a catheter;

FIGS. 5a, 5b, 5c and 5d show the various steps in setting up a prosthesis according to the invention for the treatment of stenosis on colon.

### 6. DESCRIPTION OF AN EMBODIMENT OF THE INVENTION

The general principle of the invention consists in a digestive prosthesis that is compressible and expandable in a radial direction. This prosthesis has the particularity of having an upstream collar of which the diameter is enlarged, in relation to the main body and to the downstream collar of the prosthesis. This enlarged upstream collar forms a mechanical brake that makes it possible to maintain the prosthesis in place.

According to an alternative, when the base material constituting the prosthesis is a metal material, the absence of covering by a polymer on the upstream collar, allows the digestive tissue to be in close contact with the mesh of the prosthesis. This close contact allows the prosthesis to resist the thrust exerted by the smooth muscles surrounding the digestive tract and/or that exerted by the progression of the alimentary bolus. On the other hand, the presence of the covering constituted by a polymer film on the downstream collar, and possibly on the main body of the prosthesis, makes it possible not only to facilitate the removal of the prosthesis, but also to obstruct via contact the digestive fistulae located in the digestive stenosis.

According to another alternative of the invention, when the base material constituting the prosthesis is a biodegradable polymer material, the prosthesis according to the invention is resistant to the phenomenon of migration. It can as such resist migration by dilating the stenosis until it is fully degraded.

Another advantage of the invention is to propose an upstream collar of which the wall forms an angle α with the longitudinal axis of the body of the prosthesis that is largely greater than what is currently practiced. This strong angulation, in relation to the current practice, makes it possible to form a more effective mechanical brake.

For the understanding of the example that shall follow, the values relating to the outside diameters and to the lengths are indicated for the prosthesis in its functional position, i.e. when it is fully deployed.

6.1 Manufacture Of Prostheses According To The Invention

The prostheses according to the invention are manufactured according to any method well known to those skilled in the art. The method for manufacturing such prostheses is not the subject of this application. In short, the wires of biodegradable polymer material or of metal material, for example with shape memory, are combined together in order to form a meshing. The meshing is then laser cut in order to obtain a clean and precise cut of the wires. A portion of the mesh obtained as such is then conformed in order to obtain the shape of the prosthesis.

6.2 Digestive Prosthesis Intended To Be Implanted In A Patient.

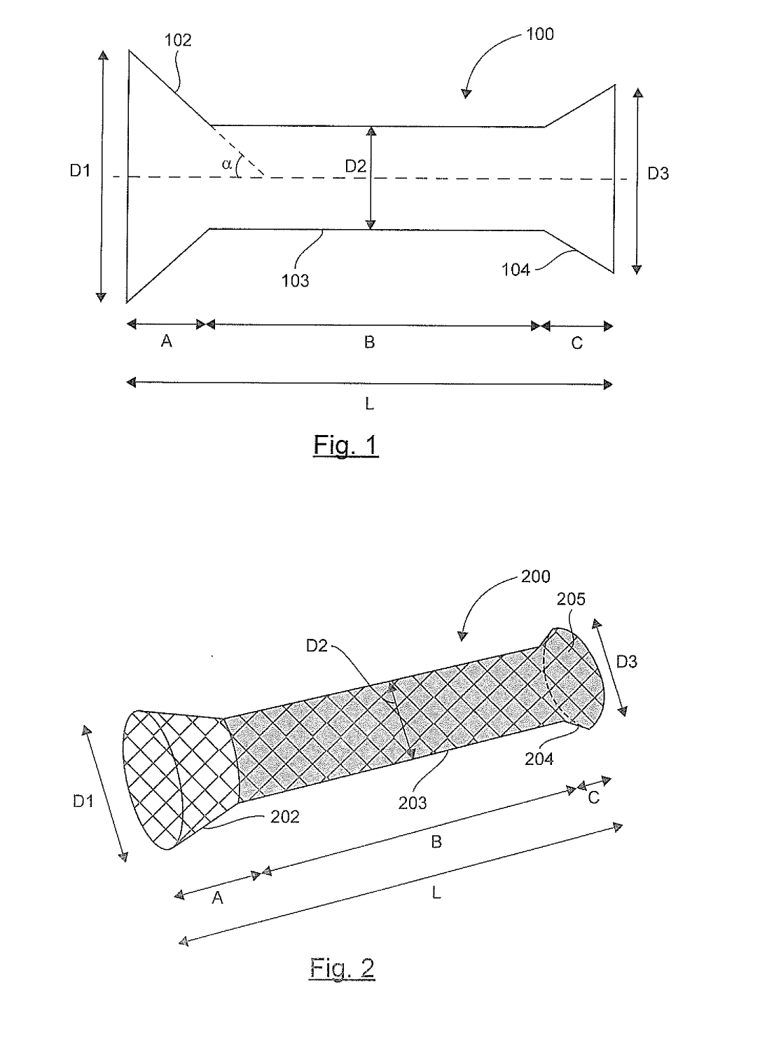

In relation with FIG. 1, the diagram of a profile view of the prosthesis according to the invention is shown, with the prosthesis being shown fully deployed. The prosthesis 100 has a generally tubular shape, with a mesh structure (not shown in FIG. 1). It is comprised of three main portions: a tubular body 103 and two collars 102, 104 arranged on either side of the tubular body 103. Each of the collars 102, 104 has a frustoconical shape, flared towards the exterior, as a profile view, when it is deployed.

The upstream collar 102 forms an angle alpha (α) with the longitudinal axis of the main tubular body 103 of the prosthesis, with the angle α being between 30 degrees and 45 degrees. In order to facilitate the understanding of the invention, the longitudinal axis is shown as a dotted line in FIG. 1. This upstream collar 102 has an end diameter D1 between 30 and 50 mm. Preferentially, the upstream collar 102 has an end diameter of at least 40 mm and forms an angle α between 30 degrees and 45 degrees with the body of the prosthesis. Preferably, the angle α is between 33 degrees and 45 degrees. It is important that this collar 102 be of the largest diameter possible, while complying with the constraints of inserting into a catheter as well as the anatomical dimensions of the digestive tract. Indeed, it is not desirable that the excessive diameter of the collar 102 cause suffering to the patient, or even that it damage the wall of the digestive tract. It is also important that the angle α that the wall of the upstream collar 102 forms with the body of the prosthesis be between 30 degrees and 45 degrees. The length A of the upstream collar 102 will be calculated according to the end diameter and the angulation that is desired to be obtained for this upstream collar.

The tubular body 103, which preferably has the shape of a cylinder of revolution, has a length B and an outside diameter D2. This length B is between 20 mm and 80 mm. Preferably, the length of the tubular body is between 24 mm and 60 mm. The outside diameter D2 of the body 103 is between 15 and 25 mm.

The downstream collar 104 has a length C between 15 mm and 25 mm, and an end diameter D3 between 25 mm and 32 mm.

Advantageously, the free ends of the upstream 102 and downstream 104 collars are rounded, in order to prevent injuring or irritating the wall of the digestive tract.

As the thickness of the prosthesis is equal to that of the wires of the material that comprises it, the inside diameters of the different portions comprising the prosthesis are slightly less than that of their outside diameter. The total length of the prosthesis L is the sum of the lengths A, B and C.

According to a first alternative, the prosthesis is made in a mesh constituted of interlacings of metal wires preferably made of a shape-memory alloy. This meshing is not covered with any covering, with the metal left bare. According to a second alternative, the prosthesis is made of a biodegradable polymer material. According to each of these alternatives, the prosthesis can then be partially covered by a film made of polymer material, on the surface of its downstream collar, but also on the surface of its main tubular body.

The mesh structure, or grid structure, allows the prosthesis to come into close contact with the digestive tissue, causing friction. This mesh structure furthermore makes it possible to obtain a prosthesis that is compressible and expandable in a radial direction, in relation to the longitudinal axis of the body of the prosthesis. The dimensions of the prosthesis will be chosen according to the application for which the prosthesis is implemented and in particular according to the anatomical dimensions of the organ to be treated.

6.3 Partially Covered Metal Digestive Prosthesis Intended To Be Implanted In A Patient.

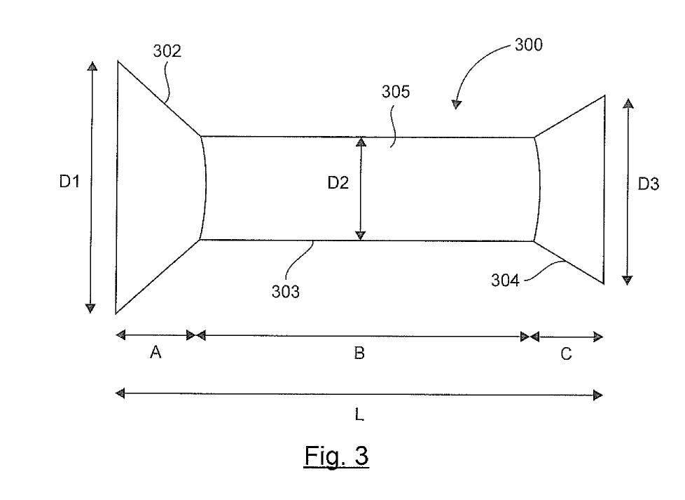

In relation with FIG. 2 a preferred embodiment of the prosthesis according to the invention is shown. The prosthesis 200 has a mesh structure. This meshing is made from nitinol, which is a nickel and titanium alloy. This material is particularly flexible and has the particularity of recovering its shape after having been compressed. Thanks to the mesh structure and to the mechanical characteristics of the nitinol, the prosthesis can be compressed in a catheter. The removal of the catheter surrounding the prosthesis allows the latter to be deployed and to return to its initial shape very rapidly.

The prosthesis 200 is constituted:

of an upstream collar 202, having a frustoconical shape,

of a body 203 having the shape of a cylinder of revolution, and

of a downstream collar 204 also having a frustoconical shape.

The upstream collar 202 has a diameter D1 of 40 mm in diameter and forms an angle α with the longitudinal axis of the prosthesis equal to 33 degrees. For clarity reasons, the angle α is not shown in this figure. The body of the prosthesis 203 has a length B of 44 mm and a diameter D2 equal to 20 mm. The downstream collar 204 has a length C of 18 mm and an outside diameter D3 of 26 mm. The collar 202 is left uncovered, while the body 203 and the downstream collar 204 are covered by a silicone polymer film 205, covering the outside surface of the entire body of the prosthesis. The presence of the silicone film 205 on the surface of the body 203 and of the collar 204 makes it possible not only to facilitate the removal of the prosthesis, but also to prevent it from being incarcerated in the mucous membrane. The absence of covering by the polymer film on the upstream collar 202 allows the prosthesis to reversibly adhere to the intestinal wall. Indeed, as the prosthesis is deployed in the intestine, the walls of the prosthesis are in contact with the intestinal mucous membrane. The absence of a covering on the surface of the mesh constituting the prosthesis prevents it from sliding against the wall. It furthermore allows the digestive tissue to colonise the meshing of the prosthesis, assisting in the attaching of the latter upstream of the stenosis. This therefore contributes to preventing, or at least limiting, the migration of the prosthesis within the digestive tract. In this way, the anti-migration method constituted by the angulation of the collar, the enlarged diameter and the absence of covering of the upstream collar make it possible to have a controlled dilatation duration that is long enough to prevent stenotic recurrence.

6.4 Partially Covered Digestive Prosthesis Made Of Polydioxanone Intended To Be Implanted In A Patient.

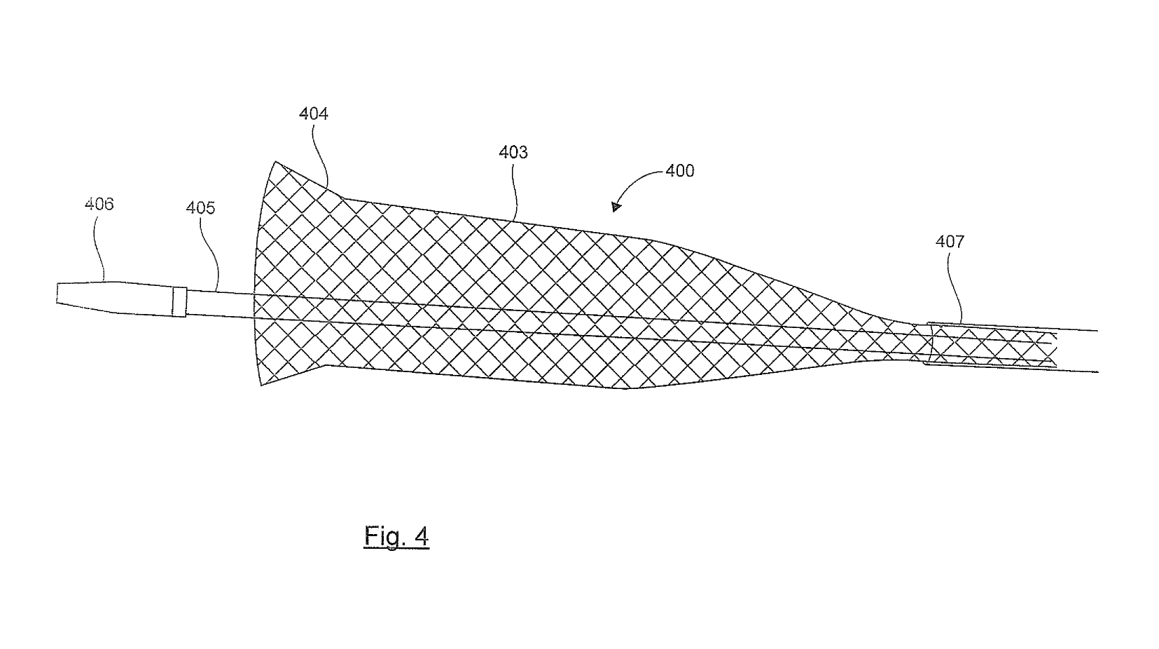

In a particularly advantageous alternative to the invention, in relation with FIG. 3, a prosthesis is described made of biodegradable polymer material having a mesh structure (not shown). This meshing is made of polydioxanone. Thanks to the mesh structure, the prosthesis can be compressed in a catheter. The removal of the catheter surrounding the prosthesis allows the latter to be deployed and recover its initial shape very quickly.

The prosthesis 300 is constituted:

of an upstream collar 302, having a frustoconical shape,

of a body 303 having the shape of a cylinder of revolution, and

of a downstream collar 304 also having a frustoconical shape.

The upstream collar 302 has an end diameter D1 of 40 mm in diameter and forms an angle α with the longitudinal axis of the prosthesis equal to 33 degrees. For clarity reasons, the angle α is not shown in this figure. The body of the prosthesis 303 has a length B of 44 mm and an outside diameter D2 equal to 20 mm. The downstream collar 304 has a length C of 18 mm and an end diameter D3 of 26 mm. The upstream collar 302 and the downstream collar 304 are left uncovered, while the body 303 is covered by a silicone polymer film 305, covering the outside surface of the prosthesis. The absence of a covering at the surface of the mesh of the upstream collar 302, combined with its enlarged diameter and the strong angulation in relation to the longitudinal axis of the prosthesis, prevents it from sliding against the wall. This friction participates in the keeping in place of the prosthesis in the digestive tract of the patient and therefore contributes to preventing, or at least greatly limiting, the migration of the biodegradable prosthesis within the digestive tract. In addition, the absence of a covering on the upstream collar allows for digestive tissue to be introduced into the orifices created by the meshing constituting the prosthesis. This physiological and normal phenomenon makes it possible to retain the prosthesis on the stenosis. As such, the duration of the dilation of the stenosis is perfectly controlled and the complications linked to the migration of the prosthesis (difficult extraction, risk of perforation, etc.) are prevented.

In addition, covering the main tubular body of the prosthesis by a polymer film makes it possible to obstruct any fistulae located in the stenosis.

6.5. Insertion Of The Partially Covered Digestive Prosthesis Intended To Be Implanted In A Patient Suffering From A Short Stenosis On Colon, By The Distal Release Method.

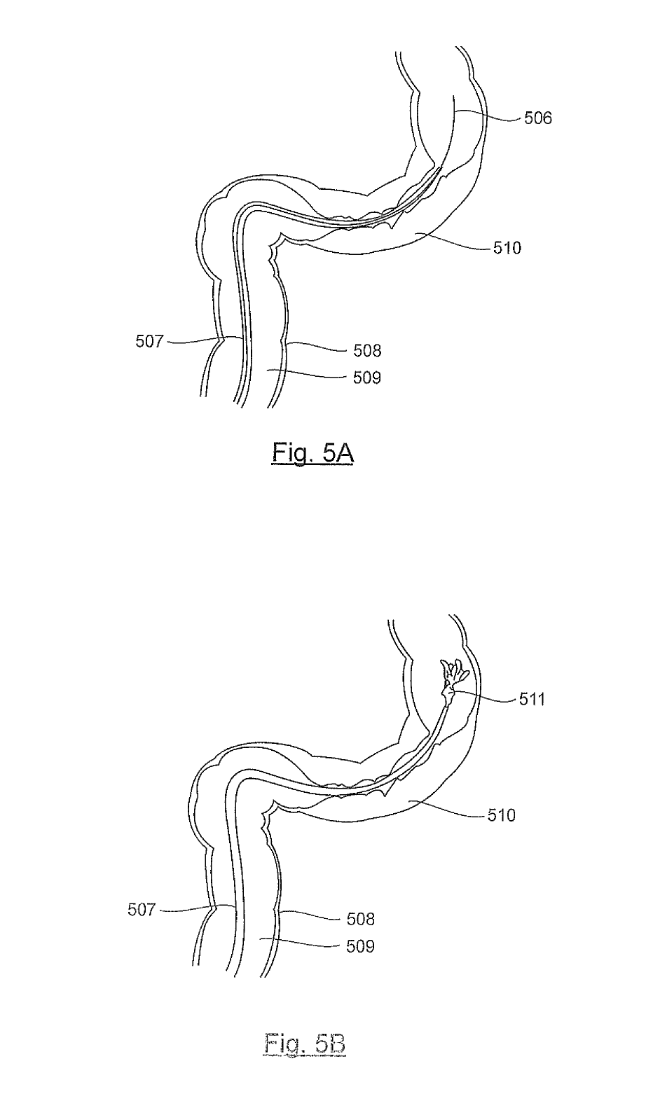

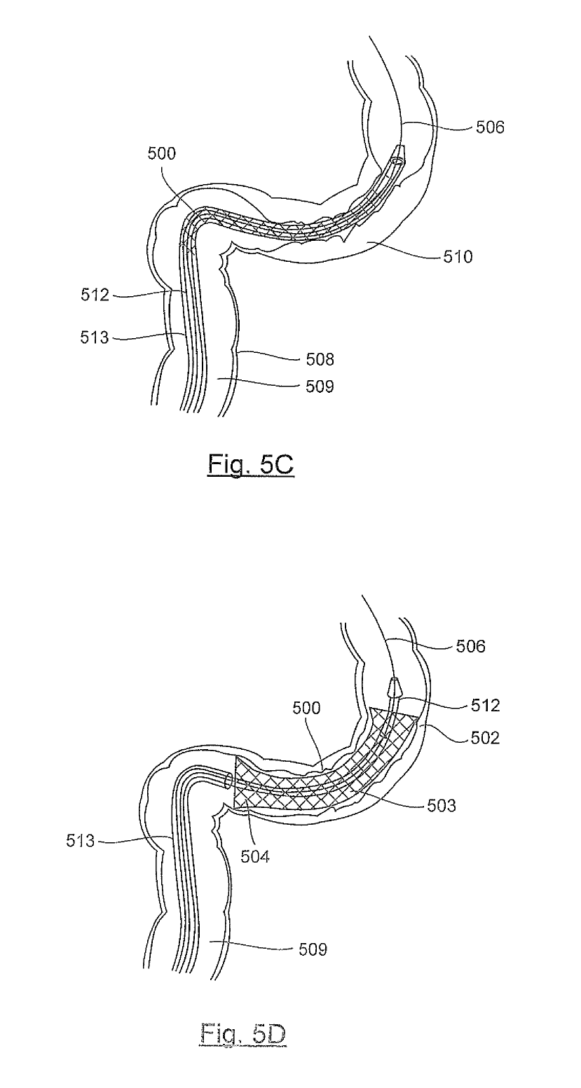

In relation with FIGS. 4 and 5A-5D the implanting of a prosthesis is described, according to example 6.3 or 6.4, in a patient suffering from a short stenosis (length <5 cm), on the anastomosis consecutive to an ileo-colic resection for Crohn's disease. This prosthesis is not covered on the upstream collar. The body of the prosthesis and/or the downstream collar can be covered by a polymer material, preferably made of silicone.

As shown in FIG. 5A, traditional endoscopy is first conducted, according to any method well known to those skilled in the art, in order to view the location of the stenosis 510 on the intestine 508 of the patient. Once the stenosis 510 is located, a flexible guide wire 506 is introduced into the intestinal lumen 509, through the lumen of the endoscope 507. The guide wire 506 is conveyed in such a way as to cross through the stenosis 510, i.e. that the guide wire passes through all of the zone narrowed by the stenosis until arriving in an area of the intestine that is free of stenosis. In this guide, a catheter is slid upstream of the stenosis and the guide is removed for opacification.

The iodinated contrast product 511, for example Telebrix®, is injected in the upstream lumen of the intestine via the catheter, the endoscope always being downstream of the stenosis, as diagrammed in FIG. 5B. This contrast product is used to view the upstream in order to correctly locate the locations of the stenosis, and the correct positioning of the endoscope and of the catheter containing the prosthesis par radioscopy.

The catheter 513 containing the prosthesis 200 is introduced into the working channel of the endoscope, still in place downstream of the stenosis, and passed under endoscopic and radiological control through the lumen of the intestinal stenosis via guide 509, as shown in FIG. 5C. This catheter contains an inner duct 512, inside of which can circulate the guide wire 506. The prosthesis 500, identical to the prosthesis 200 or the prosthesis 300, is compressed between the walls of the catheter 513 and the walls of the conduit interne 512. This prosthesis 500 comprises an upstream collar 502, a main body 503 and a covered downstream collar 504. The catheter—prosthesis—inner duct unit is pushed into the intestinal lumen 509 of the patient, until it reaches the stenosis 510. The unit is pushed in such a way as to position the prosthesis 500, by having it exceed either side of the stenosis 510. As diagrammed in FIG. 5D, the catheter 513 is then removed in order to allow the prosthesis 500 to be deployed in the intestine of the patient. This immediate deployment is made possible by the mesh structure and the use of a shape-memory material. The wire 506 and the inner duct 512 surrounding the wire are then removed to leave only the prosthesis 500 on the stenosis 510.

FIG. 4 shows an enlarged and diagrammed view of the prosthesis according to the invention during the removal of the catheter, with this withdrawal making it possible to release the prosthesis on the stenosis. Such as shown in this figure, the prosthesis 400, identical to that described in FIG. 2 or 3, is partially contained in a catheter 407. This diagram makes it possible to better observe the phenomenon of deployment of the structure made of mesh, as soon as the prosthesis is clear of the engagement of the catheter. A guide wire 405 circulates in the prosthesis 400, substantially parallel to the longitudinal axis of the latter. The guide wire 405 is flexible and is used to introduce then guide an endoscope or the catheter 407 containing the prosthesis. The distal end 406 of this guide wire 405 is lubricated in order to facilitate inserting it and it conveyance in the digestive tract of the patient. “Distal end” refers to the end that is inserted into the body of the patient. This characteristic of the guide and its flexibility make it possible to prevent the formation of lesions or downstream perforation by having allowed for the catheterization of the upstream intestine over a sufficient length by the operator.

The prostheses are then removed after 7 days, with the stenosis being stabilised in its dilatation by the prosthesis. The extraction is conducted in the following way: a colonoscopy is again conducted as indicated hereinabove. The coloscope is mounted on the stenosis which leaves visible the downstream collar of the prosthesis, still in place in its stenosis which is now dilated. The two neighbouring lassos at 3 h and 9 h on, the edges of the downstream collar are seized by an endoscopic clamp and towed toward the endoscope making it possible to close this collar like a handbag and deforming it like a missile. This makes it possible to draw this collar deformed as such via the clamp downwards, guiding it in contact with the endoscope until descending outside of the patient. It can sometimes be useful to use a lasso inserted in the middle of the inside of the body of the prosthesis in order to better tow the whole, always using a clamp; with the central lasso allowing for an invagination of the prosthesis in its centre, preventing by the same mucous membrane lesions when towing the whole.

6.6. Clinical Trial In Two Adult Patients Suffering From Short Stenosis On Colon.

Two prostheses according to the invention were implanted in two patients, both afflicted with Crohn's disease, aged 36 years and 56 years. Each of these patients underwent ileo-colic surgical resection following the development of their disease. Short stenoses, less than 5 cm, developed on the anastomosis of these resections. One of the patients had a recurring stenosis, 6 months after endoscopic dilatation.

As described in point 6.3, the upstream collar of each of the prostheses is left uncovered and has an outside diameter of 40 mm and forms an angle α of 33 degrees with the longitudinal axis of the prosthesis. The downstream collar of each of the prostheses is covered by a silicone film and has an outside diameter of 26 mm and a length of 18 mm. The body of the prosthesis measures 44 mm in length for an outside diameter of 20 mm, and is also covered by a silicone film. The prostheses were implanted as described in point 6.5, without any complication. The prostheses were then removed after 7 days. After this period, no early migration was observed, i.e. each of the patients retained their prosthesis and it did not move in the intestine. In both cases, the sub-occlusive syndrome ceded immediately after the implanting of the prosthesis. Moreover, although a resistance is generally observed during the removal of a prosthesis that has, for example, its two collars that are not covered, the removal of the prosthesis according to the invention unfolded perfectly.

The occurrence of a recurrence was monitored every month by the clinic and medical imagery. No recurrence was observed in the 3 months that followed the removal of the prosthesis. No residual stenosis or dilatation was observed in these patients.

7. Conclusions

The prosthesis according to the invention therefore makes it possible to treat short stenoses, regardless of their aetiology. The combination of the enlarged diameter and the absence of covering of the upstream collar by any material makes it possible to prevent, or at the very least substantially reduce, the problem of early migration, which is a major risk with the prostheses currently on the market. The presence of a covering with a polymer material of the downstream collar makes it possible to facilitate the removal of the prosthesis and to prevent injuring the digestive wall of patients during this manipulation. The possible presence of a covering by a polymer on the body of the prosthesis furthermore makes it possible to facilitate the removal of the prosthesis, and even limit the phenomenon of incarceration. In addition, the strong angulation of the upstream collar, in relation to the prostheses of prior art, constitutes an extremely effective mechanical brake, without however damaging the inner wall of the digestive tract or causing pain for the patient.

As such, the disadvantages linked to the use of expandable prostheses for the treatment of short stenoses are now able to be eliminated thanks to the prosthesis according to the invention, or at the very least to be greatly reduced.

The absence of a covering by a polymer on at least the upstream collar could lead one to believe that there was a risk of incarceration at this level. However, the in vivo trials showed that this phenomenon has never been observed.

Particularly, a prosthesis according to the invention made from a metal base material and having an enlarged upstream collar and not covered by any material, as well as a main body and a downstream collar covered by a polymer material, makes it possible to resolve both the problems of migration and of incarceration observed. The presence of the polymer film furthermore makes it possible to obstruct any fistulae that are sometimes produced, in particular following sutures.

Moreover a prosthesis according to the invention made from a metal base material and having an enlarged upstream collar and not covered by any material, a main body not covered by any material and a downstream collar covered by a polymer material would find its interest for the palliative treatment of stenoses in patients suffering from colorectal cancer. Such a prosthesis is also more flexible, due to the low portion of its surface covered by a polymer material. It therefore makes it possible to solve the problem of perforation of the digestive wall.

Finally, according to a second alternative of the invention a prosthesis made from a base material constituted by a biodegradable polymer material makes it possible to solve the problem of migration, the problem of perforation and possibly, the problem of incarceration. Furthermore, it does not produce any waste and its environmental impact is lower than that of metal prostheses.

As such, the disadvantages linked to the use of expandable prostheses for treating short stenoses are now able to be totally or partially eliminated, or at the very least be substantially reduced.

Note that other embodiments of the invention can be considered. In particular, it is possible to add opaque radio markers or fluorescent markers on the surface of the prosthesis in order to allow it to be seen in radioscopy or fluoroscopy. It can also be provided to pre-equip the prosthesis according to the invention with a guide wire, circulating substantially parallel to the longitudinal axis of the prosthesis, in order to facilitate its inserting and its positioning through the stenosis. Finally, it can be provided to associate anti-reflux valves for oesophageal applications.

It can also be considered to cover the prosthesis according to the invention, or to include in the polymer material covering, at least one active ingredient, more preferably a therapeutic active ingredient. As such the therapeutic potential of the prosthesis according to the invention will be increased.

In addition, although the clinical trials exposed hereinabove relate only to the treatment of colonic stenoses, these prostheses are entirely suited for treating oesophageal, pyloric, tracheobronchial, etc. stenoses. It is sufficient, to do so, to adapt the relative proportions of the various portions of the prosthesis so that it can be inserted into the anatomical structure that one wishes to treat.

A distal release has been described in this application of the prosthesis and via abdominal endoscopy in the digestive tract of the patient, namely that the prosthesis was introduced into the digestive tract of the patient through the anus (i.e. endoscopy via the abdominal route). Distal and proximal are expressed in relation to the endoscopic vision: the prosthesis is deployed by its end (collar) that is the farthest away from the endoscope as distal release and by its end (collar) that is closest to the endoscope as proximal release. It is understood that the prosthesis according to the invention can also be placed in a patient by upper-route endoscopy, by passing through the mouth of the patient. By this route, the release method (i.e. deployment of the prosthesis when the catheter carrying it is immobilised) can be distal, which is the most frequently used case in digestive endoscopy, or proximal. If the upper route is used with distal release, the technique is not different from what has been described hereinabove as abdominal route: the catheter is pulled and the prosthesis is deployed via its distal end in relation to the endoscope. On the other hand, in the upper route with proximal release, the catheter will be pushed from upstream to downstream in order to release the prosthesis. Once the prosthesis is fully deployed, it is sufficient to remove the empty catheter with its guide. Regardless of the type of release with upper route, the prosthesis must be manufactured in such a way that the anti-migration collar is always as close as possible to the endoscope in its conveying catheter so that it is it that is upstream of the stenosis to be treated, in relation to the direction of flow and digestive motricity.

1. A prosthesis that is compressible and expandable in a radial direction, said prosthesis configured to be implanted in the digestive tract of a patient, said prosthesis being made from a base material and comprising:
a downstream frustoconical collar having an end diameter D3;
a main tubular body having a diameter D2; and
an upstream frustoconical collar having an end diameter D1;
where said upstream frustoconical collar is not covered by any other material and the end diameter D1 is greater than the diameter D2 of said main tubular body and greater than the end diameter D3 of said downstream collar, and said downstream frustoconical collar is covered or constituted, fully or partially, by at least one polymer material.

2. The prosthesis according to claim 1, characterised in that the prosthesis has a mesh structure.

3. The prosthesis according to claim 2, wherein said base material is a metal material chosen from stainless steel, titanium, chromium, cobalt or a combination of a least two of these materials, and said downstream frustoconical collar is covered by at least one polymer material.

4. The prosthesis according to claim 1, characterised in that said base material is at least one biodegradable polymer material and said downstream frustoconical collar comprises said polymer-base material.

5. The prosthesis according to claim 4, wherein said at least one biodegradable polymer material is chosen from among polydioxanone (PDS), polylactic acid (PLA), poly-L-lactic acid (PLLA), polyglycolic acid (PGA), or ε-caprolactone.

6. The prosthesis according to claim 4, wherein said at least one biodegradable polymer material is polydioxanone.

7. The prosthesis according to claim 4, wherein said mesh structure comprises a woven or knitted multifilament or monofilament threads of said at least one biodegradable polymer material.

8. The prosthesis according to claim 1, characterised in that said main tubular body is covered, fully or partially, by at least one polymer material.

9. The prosthesis according to claim 3, characterised in that said at least one polymer material is chosen from the group comprised of polyurethane, polyvinyl chloride, urethane, silicone, polyamide, polyester, fluorine resin, polytetrafluoroethylene or a combination of at least two of these materials.

10. The prosthesis according to claim 1, characterised in that a wall of said upstream frustoconical collar forms an angle alpha (α) between 30 degrees and 45 degrees in relation to a longitudinal axis of said prosthesis.

11. The prosthesis according to claim 1, characterised in that said prosthesis has a total length between 60 mm and 120 mm.

12. The prosthesis according claim 1, characterised in that said upstream frustoconical collar has the end diameter D1 between 30 mm and 50 mm.

13. The prosthesis according to claim 1, characterised in that said main tubular body comprises a length between 20 mm and 80 mm and the outside diameter D2 between 15 mm and 25 mm.

14. The prosthesis according to claim 1, characterised in that said downstream frustoconical collar has a length between 15 mm and 25 mm and the end diameter D3 between 25 mm and 32 mm.

15. The prosthesis according to claim 1, characterised in that the prosthesis comprises at least one radiopaque marker.

16. The prosthesis according to claim 1, wherein said polymer material is covered in or includes at least one active ingredient.
